(19) United States
(12) Patent Application Publication
(10) Pub. No.: US 2004/0102380 A1
(43) Pub. Date: May 27, 2004

(54) Method for continuous, automated blending of solutions from acids and bases

(76) Inventors: Scott P. Fulton, Middleton, WI (US); Peter G. Brown, Newton, MA (US)

Correspondence Address: GTC BIOTHERAPEUTICS, INC., 175 CROSSING BOULEVARD, SUITE 410, FRAMINGHAM, MA, 01702, US

(21) Appl. No.: 10/715,701

(22) Filed: Nov. 18, 2003

Related U.S. Application Data
(60) Provisional application No. 60/427,316, filed on Nov. 18, 2002.

(51) Int. Cl. A61K 38/38; C12P 21/02

(52) U.S. Cl. 514/12; 435/69.1

(57) ABSTRACT

The present invention relates to an improved method to process, purify and/or produce biopharmaceuticals or other products involving automated blending of pH buffered solutions from water and common stocks of concentrated acids and bases, and other components. This approach reduces the cost and complexity of the solution preparation systems required for producing these solutions under aseptic or sterile conditions, and reduces the material costs of the solutions themselves. This approach is particularly beneficial to use with continuously-produced feedstocks and with continuous separation operations.

### FIELD OF THE INVENTION

The present invention relates to an improved and more efficient method of producing aqueous buffers and other aqueous solutions used for various unit operations such as chromatography in the processing of biopharmaceuticals or other applications by utilizing continuous generation from common stocks of concentrated constitutive acids and bases, as well as salts and other needed reagents.

### BACKGROUND OF THE INVENTION

The present invention is directed to a method of producing solutions which require pH-controlled buffers either for product processing operations or as the final product. These processes or products have in common the need to control pH, which is done through the use of a buffer compound containing ionizable groups, and adjusting the pH of the solution to within approximately 1 pH unit above or below the pKa of the ionizable groups. In this pH range, the ionization equilibrium of the ionizing groups has a buffering effect, making the pH of the solution reasonably stable to small changes in pH from chemical reactions to which it may be exposed that add or remove hydrogen ions from the solution. In current industry practice, these pH buffer solutions are usually created by making an aqueous solution of a purified salt form of the buffering compound, adding any additional solution components required for the application (such as other salts, surfactants microbial inhibitors, and the like) and then adjusting the pH of the solution up or down by the controlled addition of either acid or base (often HCl or NaOH) as required. The buffering compound and additives are most often in the form of dried (often crystalline) salts, which are relatively expensive. The acid or base forms of the buffering compound are often supplied as a concentrated liquid, and are most often substantially less expensive than the corresponding dried salt.

Applications for pH buffered solutions include all of the unit operations used in production and downstream purification of biopharmaceuticals, including those produced by fermentation of microbes, fungus or yeast, mammalian or insect cell culture and transgenic animal and plant sources. The unit operations which use pH buffered solutions include filtration, centrifugation, precipitation, crystallization and chromatography. Chromatography operations in particular utilize different pH buffered solutions for loading the column, washing, eluting the product, regenerating, and re-equilibrating the column. Every unit operation is achieved in discrete sub-batches or cycles, with a product batch comprised of one or more unit operation cycles. Other applications for the invention might include products which themselves are pH buffered solutions. Examples of such products include ophthalmic solutions and infusion solutions.

In these applications for this invention, the final use of the buffered solutions often requires that the solutions be aseptic, and in some cases sterile. The final blended buffer solution is often quite supportive of microbial growth. Practical production, handling and storage of aseptic or sterile solutions requires very careful, specialized and expensive design and construction of all the equipment which contacts the solution. In addition, the equipment must be subjected to rigorous clean-in-place (CIP) procedures following usage to insure no chance of microbial contamination being present for the next batch, and may also require steam-in-place (SIP) procedures to insure sufficiently clean conditions. The water used for these applications is produced to very high purity requirements (most often water-for-injection or WFI), and is costly to utilize. These requirements for aseptic or sterile system make both the capital and operating costs of such processes very high.

The concentrated acids and bases, and in many cases other ingredients in highly concentrated forms (such as salts) do not themselves support microbial growth. In fact, the highly concentrated acids and bases are often themselves used as the primary cleaning solutions for CIP operations, because of their ability to at least partially sanitize process systems. Thus the storage tanks and distribution systems for these ingredient feeds in the present invention do not necessarily need to be designed, constructed and operated to meet aseptic or sterile standards, and can thus be far less expensive and much simpler.

In many modernized plants tasked to the production of biopharmaceuticals, the systems designed for unit operations require both large capital outlays and a large labor force. The state of the art is such that the current processes provide to the combination of multiple buffers, eluents, regenerants, and other solutions employed in the unit operations individually. The components for each of these numerous and various solutions are mixed with the appropriate pharmaceutical grade water (such as water for injection or “WFI”) in large, shared solution blending tanks. Thereafter, the resulting solution is microfiltered, tested, and transferred to individual, dedicated holding tanks before the commencement of the processing which utilizes a specific batch of a reagent. Subsequent to the usage of the batch of solution, the transfer piping system and the blending tank need to be meticulously cleaned in place “CIP” and often SIP procedures prior to the production of the next solution.

Also, according to the prior art, synchronizing the solution preparation operations to enable the equipment to be utilized well and to ensure the accessibility of all solutions when needed can amount to a substantial challenge and incurs substantial cost. In an ordinary biopharmaceutical and pharamaceutical production facility of the prior art, a significant portion of the space and capital investment is reserved for solution preparation, a distribution system, and a multitude of solution storage tanks. In addition, with batch-wise blending, the span of scales that can be managed by a specific dimension of tanks and distribution systems is restricted. If the tanks are too limited in volume, they will lack the capacity required for a whole batch or cycle of production. If they are too large, the solutions will remain stationary for too long sometimes allowing inappropriate or economically undesirable chemical changes, and capital investment will be excessive for small scales, leading to a lack of commercial flexibility.

In more recent years, some biopharmaceutical production facilities have been designed using the concept of producing and storing concentrates of the solutions, which are then diluted online with the appropriate pharmaceutical-grade water at the point of use. This approach can reduce the size of the required solution storage tanks, and significantly reduce the number of times batches of solutions must be produced and the storage tanks and distribution systems cleaned. However, the number of storage tanks and the complexity of the distribution systems is not reduced with this approach. Also, the ultimate concentration factor of the storage form of the solution is limited by the solubility of the least soluble component.

As the scale of biopharmaceutical processing operations is increasing, plants are being designed and built with continuous unit operations instead of the conventional batch operations. Continuous cell culture approaches, for example, are becoming quite commonplace. Transgenic production systems are either semi-continuous (as for example with transgenic dairy animals, which produce milk 2-3 times every day) or can be treated as such (as for example with transgenic crops, which can be stored for long periods as a feed for continuous downstream processing). Increasingly, continuous downstream purification unit operations are also being developed. An example of such a unit operations is simulated moving bed or SMB chromatography.

Although maintaining batch integrity involves less difficulty to comply with the regulatory requirements of strict traceability of all procedures and materials employed in the production of a given lot of final drug product, there are disadvantages and problems to batch design. The most paramount is the inefficient utilization of equipment capacity. For a significant portion of the time, any given tank or other piece of equipment in the plant is simply waiting for the execution of the antecedent steps, for the unit operations, or for the following batch. Meticulous succession and staggering of cycles can aid in the enhancement of capacity utilization; however, the stepwise sequence within the unit operations places a restriction on this approach. There is a viable need to notably enlarge the capacity utilization, particularly for products manufactured on a relatively substantial scale (hundreds of kilograms to tons per year).

Continuous processes place particular demands upon the solution preparation systems within a production plant. Because the solutions must be supplied continuously, it is not possible to stop to clean the storage/feed tanks, produce new batches of needed solutions and then refill the tanks. Therefore, in such plants each solution must have two storage tanks with associated distribution systems—one for supply of the operation itself and a second which is being cleaned and refilled while the first is being utilized. This requirement significantly increases the cost of such facilities, and negates some of the benefits of continuous operations.

With regard to the prior art, individual patents are discussed below, U.S. Pat. No. 4,907,892 entitled “Method and Apparatus for Filling, Blending, and Withdrawing Solid Particulate Material from a Vessel” discloses a method for blending solid, particulate material with liquids to form a suspension, with an apparatus with a continuous blending unit. This method, however, neither blends solutions to create aqueous buffers nor allows for the production of biopharmaceuticals. Moreover, the apparatus contains a sensor to monitor the quantity of material in the vessel by its height or weight plus a controller that responds to the sensor for regulating the particulate material feed rate or the material withdrawal rate in order for the material supply rate and blended substance withdrawal rate to be balanced to direct the material level inside the vessel to a preferred level. In FIG. 3 of this application, in the blending unit, positive displacement chemical metering pumps are utilized to proportion the ingredient streams, entering the processing plant, not to regulate or to measure the amount of solution in the blending unit. The blend for each solution is regulated by the combination of the pump head sizes and adjustable stroke lengths.

In U.S. Pat. No. 6,180,335 entitled “Apparatus for Detecting Contamination in Food Products” the food sample is combined with a buffer solution and a blending buffer. According to the claims of this patent, the purpose of the mixing event with a buffer solution is to ultimately quantify the amount of bacterial contamination in a food sample. The claims do not disclose a method of producing pH buffered solutions themselves in a continuous or automated way. Moreover, the solution does not appear to be involved in any pharmaceutical production, but rather a diagnostic application.

In U.S. patent application Ser. No. 20020156336 entitled “Method for Continuous Detoxification of Poisonous Agent or Toxic Chemical Compound, or Soil Contaminated by Said Poisonous Agent and/or Toxic Chemical Compound” discloses a method for continuous detoxification of substances by blending of reagents with the feedstream to be detoxified, but does not contemplate or disclose the production of biopharmaceuticals.

In U.S. Pat. No. 6,186,193 entitled “Continuous Liquid Stream Digital Blending System,” this invention is directed to a method and an apparatus for continuous stream blending. The approach taught in this patent is to blend an appropriate number of small-volume “digital slugs” of fluid in a tank as a convenient way of producing a blended stream. It does not teach the specific use of blending constitutive acids and bases to produce a pH buffered solution, particularly for the use of biopharmaceuticals.

U.S. Pat. No. 6,162,392 entitled “Method and Apparatus for Super Critical Treatments of Liquids,” this invention is directed to a method to sterilize a liquid in a continuous, pressurized system consisting of de-pressurizing and cooling steps, not related to producing biopharmaceuticals. This patent utilizes pumps for controlled flow rate and increases and decreases in the temperature of a treated solution, but does not involve blending of chemicals.

In U.S. Pat. No. 5,823,669 called “Method for Blending Diverse Blowing Agents” discloses a method for continuously and precisely blending multiple gaseous or volatile liquids at low pressures, not buffering solutions.

U.S. Pat. No. 5,552,171 entitled “Method of Beverage Blending and Carbonation” discloses a method and an apparatus to procure a very precise control of the blend, but it does not involve the blending of buffer solutions for pharmaceutical purposes.

In U.S. Pat. No. 5,340,210 referred to as “Apparatus for Blending Chemicals with a Reversible Multi-Speed Pump” discloses an apparatus to blend substances with a pump for each type of chemical such as water-based and oil-based. This invention discloses multi-speed pumps which do not pertain to proportioning the ingredient streams.

The prior art (both within patents and in industry practice) teaches numerous methods of using continuous blending to produce various types of chemical solutions from mixes of solids, liquids and gases. However, the prior art does not teach a continuous, automated blending from constitutive acids and bases of pH buffered solutions used for the production of biopharmaceuticals or other products, according to the method of the current invention. Moreover, the current invention provides advances in biopharmaceutical production that allow processing of compounds, especially biopharmaceutical, on a more efficient and economically flexible basis. The invention can reduce the material costs for these products through the utilization of less expensive acids and bases rather than the more expensive dried salt forms of the buffering compounds. In addition, the current invention, according to a preferred embodiment, is much more suitable for continuous (instead of batchwise) production methods fermentation. Such production methods can be used with continuous perfusion cell culture and the production of proteins from the milk of transgenic dairy animals or from transgenic plant extracts, where the seed or plant form may provide very long term storage of the raw material, enabling continuous unit operations for purification.

### SUMMARY OF THE INVENTION

According to the current invention, the batchwise, manual blending of pH buffered solutions is improved upon through the use of an automated solution blending technique of the current invention. This method utilizes concentrated acids and bases to form the primary buffer solution, and concentrated solutions of salts, surfactants or other additives blended in to form the final solution. In a preferred embodiment of the current invention, a small number of feed solutions is used to make a variety of reagent compositions improving efficiency of operation, decreasing error, and lowering cost. Moreover, the operation may be, in a preferred embodiment, continuous.

The buffering compounds can include inorganic acids (such as phosphoric or boric acid), simple organic acids (such as acetic or citric acids), organic bases (such as tris-hydroxymethyl amino methane (TRIS), and so-called Good's buffers including HEPES, MOPS, MES, etc.). The buffering compound is usually combined with a strong base (such as sodium or potassium hydroxide), or a strong acid (such as hydrochloric) as appropriate to produce the final pH desired. The acid and base are supplied to the system as liquid concentrates, usually at a very high concentration. Other ingredients are also supplied as pure liquids or concentrated solutions. These other solution ingredients can include salts (such as sodium, potassium or magnesium chloride, sodium or ammonium sulfate,, and the like), surfactants (such as Tween), chaotropic or solvophobic agents (such as ethylene glycol, urea, sodium thiocyanate, or guanadinium hydrochloride), mild reducing agents (such as cysteine or mercaptoethanol), microbial or proteolytic inhibitors (such as thimerosol, sodium azide, and the like), precipitation or extraction agents (such as polyethylene glycol, dextran, and the like), etc.

Once the ingredients are properly loaded into the processing plant, the individual ingredients are blended. In one embodiment the ingredients are continuously blended on demand by pumping the various streams (water, acid, base and other additives) at controlled flow rates into a mixing device (static or active mixer), and the resulting pH buffered solution is then used directly and immediately in the process. Control of the pH may be implemented by placing a pH sensor downstream of the mixing point and using the value to control the relative flow rates of the acid and base streams.

In a second embodiment, the individual ingredients are pumped either simultaneously or sequentially into a small, stirred tank with sensors for pH, conductivity, temperature, and level. When this small tank is filled and mixed, the solution characteristics are reviewed (either automatically or manually) against specifications. If the results are approved, the individual solution is released. A second small buffer tank can be employed to permit time for blending and checking. This practice ensures that the same Good Manufacturing Practices (GMP) quality standards can be satisfied as with batchwise solution blending.

Utilization of this method results in reduction in cost for buffer solutions by employing concentrated buffer acids and bases instead of more expensive buffer compound salts. Moreover, a considerable reduction of costly sanitary design tankage and piping proceeds from this method. A very broad scale range is able to be accomplished without more capital expenditures. The approach used in the invention is also highly advantageous for continuous processes and unit operations. Other features and advantages of this invention will become apparent in the following detailed description of preferred embodiments of this invention, taken with reference to the accompanying drawings.

### BRIEF DESCRIPTION OF THE DRAWINGS

FIG. 1 shows a downstream processing plant with the conventional batchwise solution blending of the prior art.

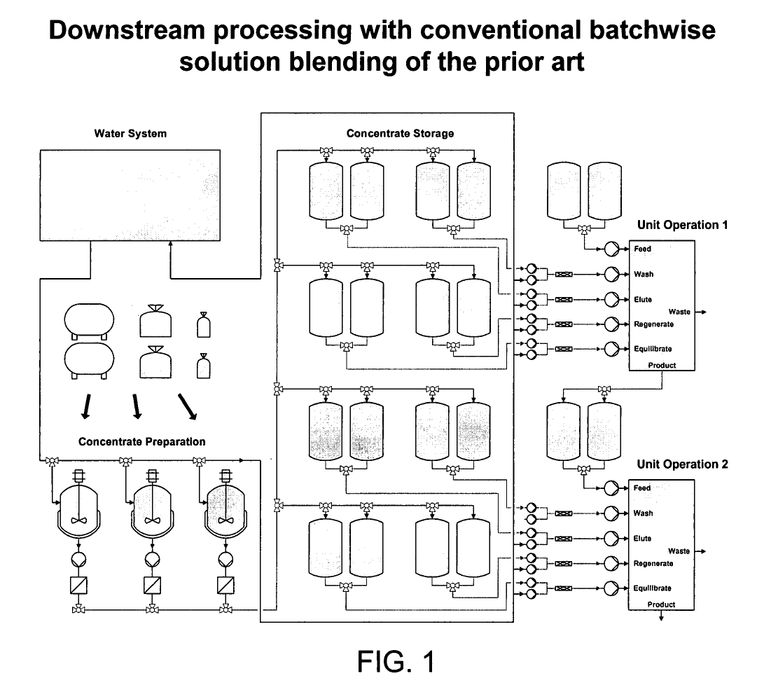

FIG. 2 shows a downstream plant demonstrating continuous solution blending from acids and bases.

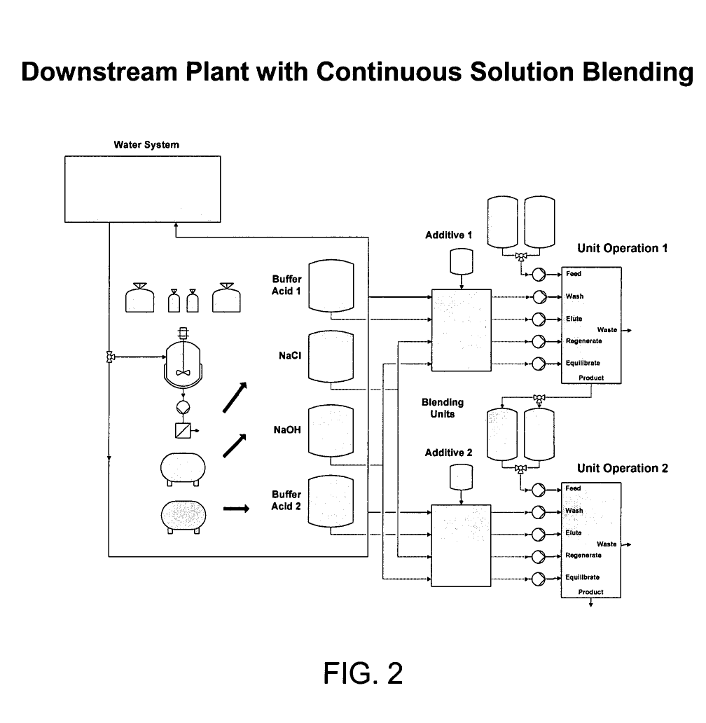

FIG. 3 shows a buffer blending unit design for direct online blending.

FIG. 4 shows a buffer blending unit according an embodiment utilizing an inline mixing tank.

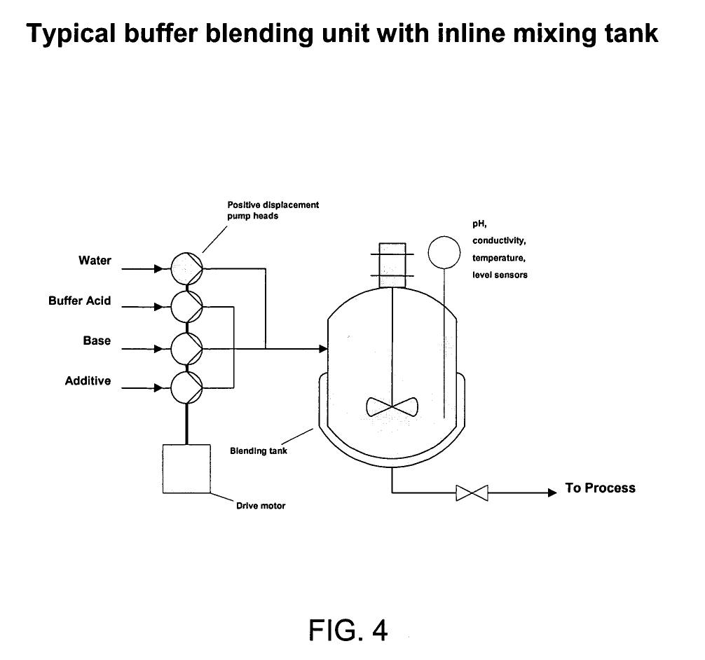

FIG. 5. shows a model of the facility elements of a typical of a biopharmaceutical production plant

FIG. 6 shows a transgenic human serum albumin process scheme.

FIG. 7. shows a chart comparing the cost of the current invention relative to conventional batchwise processing.

FIG. 8 shows human serum albumin process scheme utilizing a simulated moving bed design.

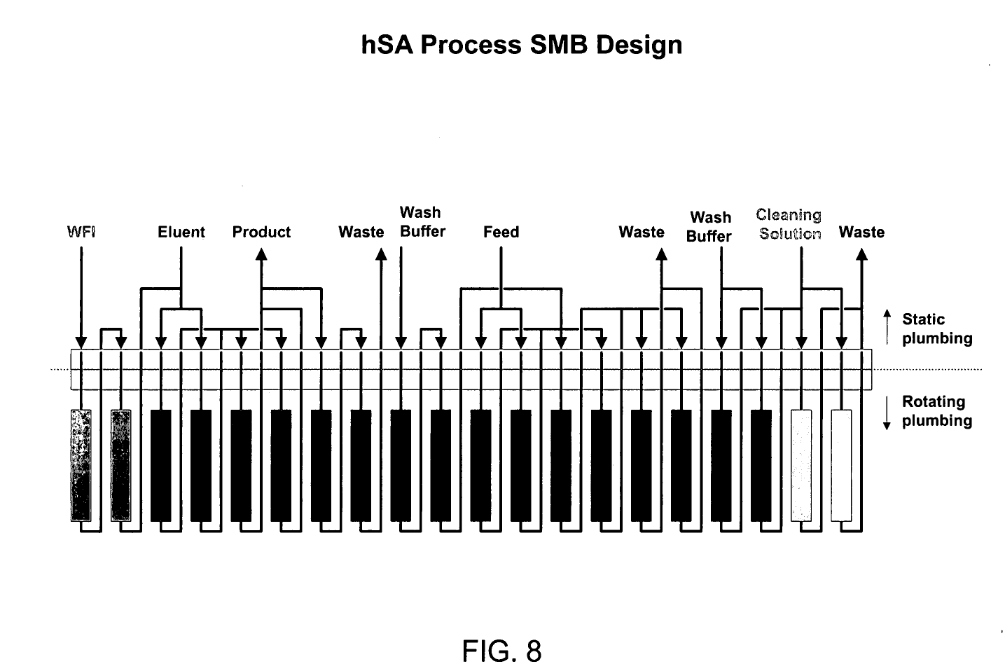

FIG. 9 shows an alternate and simplified transgenic human serum albumin process scheme.

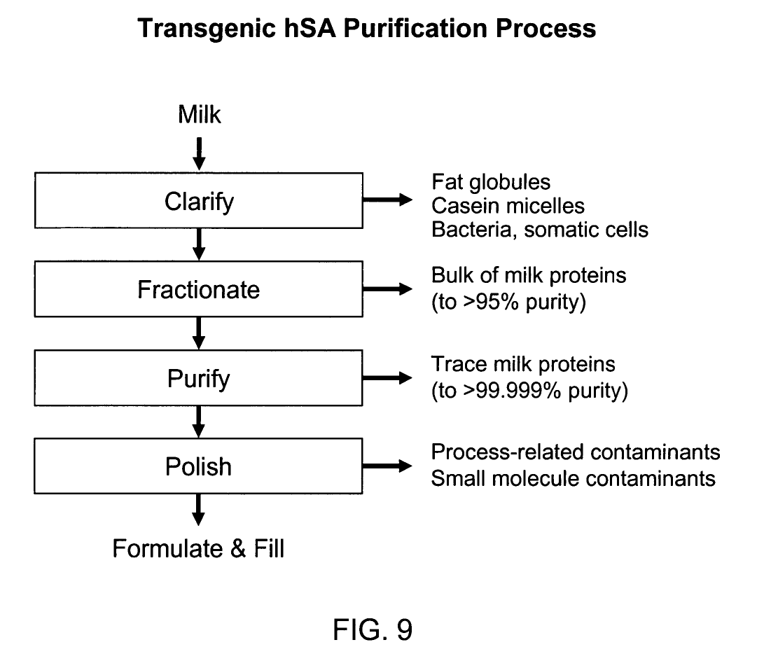

FIG. 10 shows a downstream plant demonstrating continuous solution blending from acids and bases and SMB.

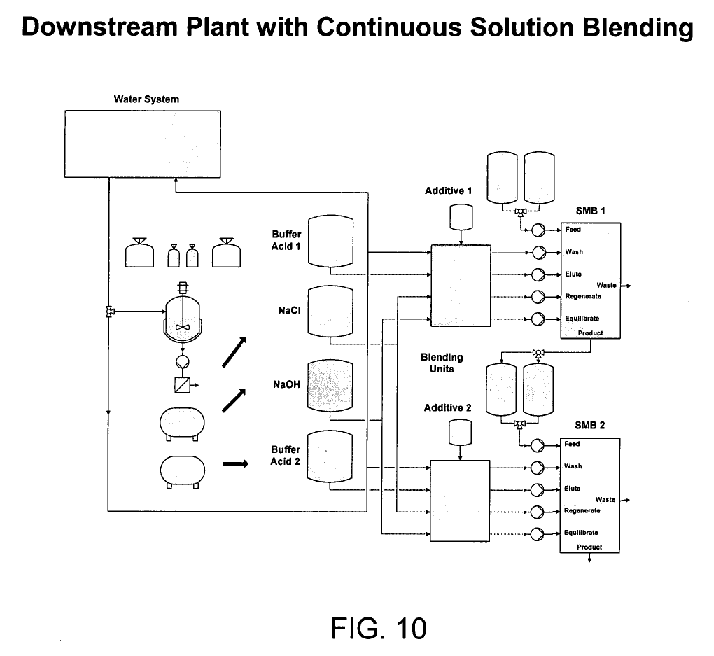

### DESCRIPTION OF THE PREFERRED EMBODIMENTS

The following abbreviations have designated meanings in the specification:

Abbreviation Key:

SMB An abbreviation for simulated moving bed chromatography.

pH A term used to describe the hydrogen-ion activity of a chemical or compound according to well-known scientific parameters.

WFI An abbreviation for water for injection.

CIP An abbreviation for cleaned in place.

GMP An abbreviation for Good Manufacturing Practices.

Explanation of Terms:

Biopharmaceutical

shall mean any medicinal drug, therapeutic, vaccine or any medically useful composition whose origin, synthesis, or manufacture involves the use of microorganisms, recombinant animals (including, without limitation, chimeric or transgenic animals), nuclear transfer, microinjection, or cell culture techniques.

Buffers

a system that acts to minimize the change in concentration of a specific chemical species in a solution against the addition or depletion of this species.

Cell Culture

general term referring to the maintenance of cell strains or lines in the laboratory.

Chromatography

any of a multitude of techniques for the separation of complex mixtures that are dependent upon the differential affinities of substances for a gas or liquid mobile medium and for a stationary absorbing medium.

Feedstream

the raw material or raw solution provided for a process or method and containing a protein of interest.

Simulated Moving Bed Chromatography

a continuous solid-liquid dissociation method that purifies two components of a feedstock. Both components are generated at a superlative yield and purity.

The method of the current invention provides an efficient process to produce pH buffered solutions that will ultimately be converted into or used as pharmaceutical products. The primary ingredients that compose a mixture are water, and a buffer acid and base at a particular concentration and in a particular ratio to produce a desired final pH. In addition, the solution may include other solution ingredients, such as salts, surfactants, inhibitors etc., see detailed listing above. The individual ingredients are blended at the point of use using an automated blending unit.

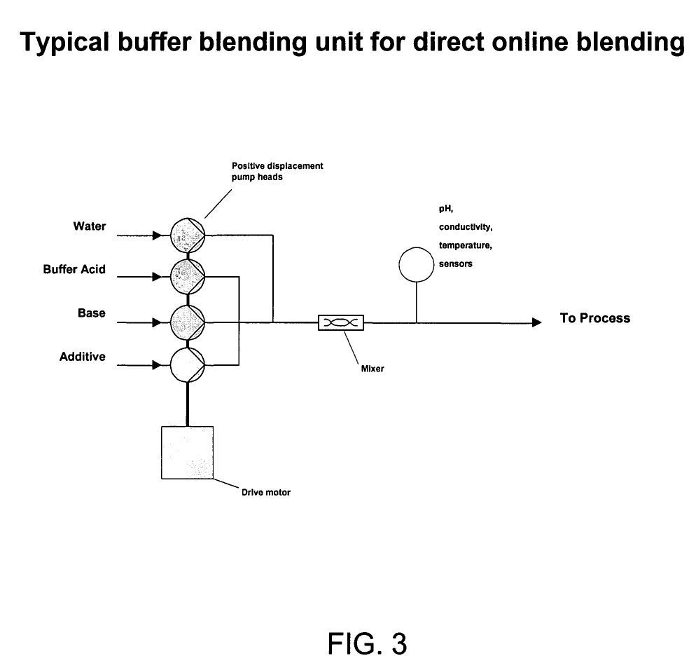

In one preferred embodiment of the invention, as shown in FIG. 3, reciprocating, positive displacement chemical metering pumps are used to regulate the flow of the ingredient streams. The precise blend for a particular solution is fixed by the combination of pump head sizes and flexible stroke lengths. The various streams are simultaneously pumped into a mixing unit of either a static or active type. If required, sensors for pH and conductivity can be placed inline after the mixer and their output utilized to control the relative ratios of the acid, base and other ingredients. In this embodiment, the solution is utilized immediately by the process being supplied.

In a second preferred embodiment, the solution ingredients (water, acid, base and any other ingredients) are metered out by pumps and mixed in a small tank. The metering operation can be done simultaneously for all ingredients (using the same type of positive displacement chemical metering pumps utilized in the first embodiment). Alternatively, the metering can be done sequentially for each ingredient, using either metering pumps or control through the use of a level sensor or load cell placed on the mixing tank. The mixing tank would be equipped with sensors for pH, conductivity, level and possibly other parameters. When the blending operation in the small mixing tank is completed, the sensor measurements would be compared to a release specification, and the solution would be released for use in the process if the specifications are met. If the solution is required to be supplied continuously to the process, two small mixing tanks could be used, one of which would supply released solution while the other is being used to blend a new tank of solution.

The first preferred embodiment of the invention is simpler and less expensive to construct, and may be truly continuous, according to a preferred embodiment of the invention. This would be the embodiment used for a large fraction of the applications. The second embodiment incorporates some of the current elements of good manufacturing practice (GMP) for pharmaceutical manufacturing, and may be required for some particularly critical process steps.

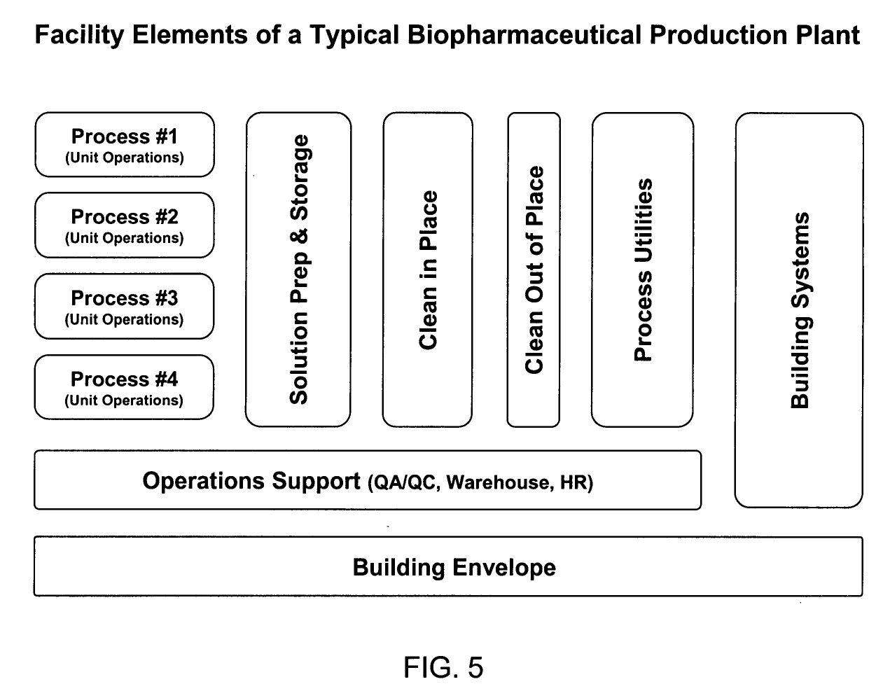

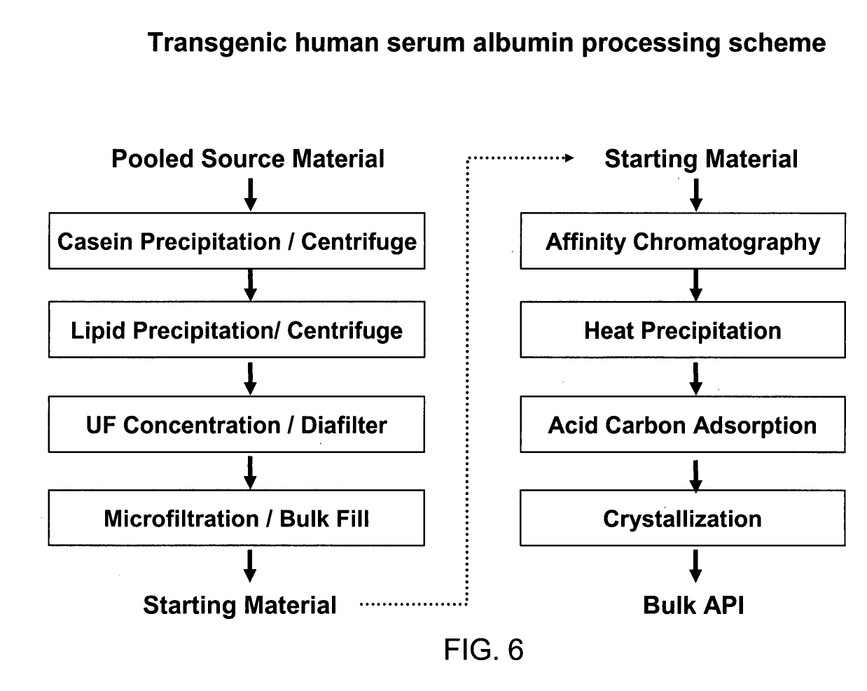

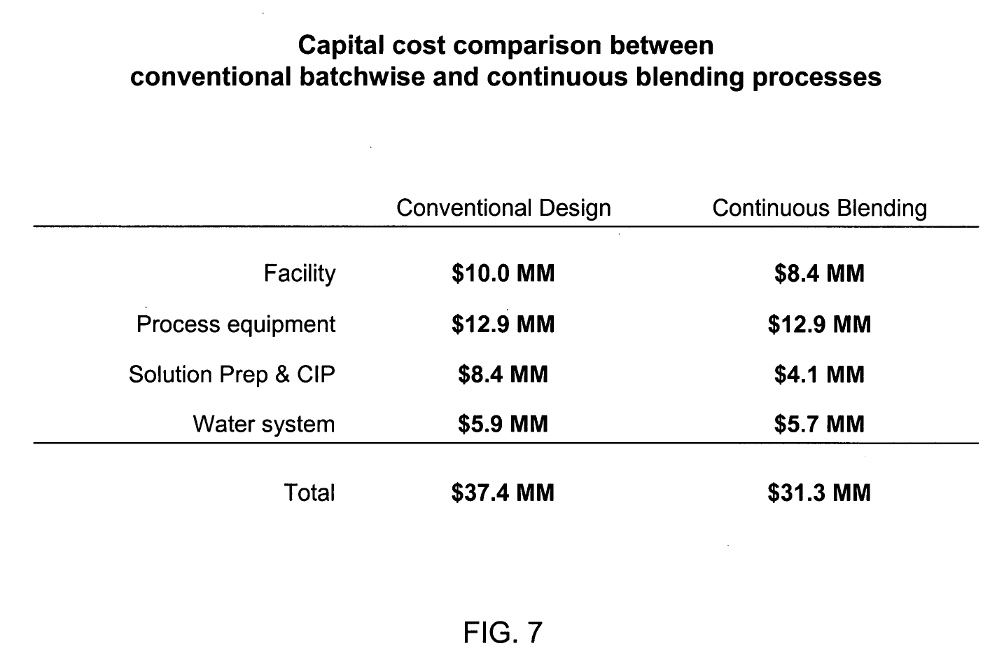

Turning to FIG. 7, the design and testing data on the human serum albumin downstream purification process shown in FIG. 5 were used as input to a detailed process cost modeling software system (Paradigm One, Applied Process Technologies, Wilmington, Mass.). The software package estimates detailed capital and operating costs based upon specific process parameters, selected equipment, utility and space requirements, etc. For this model, a facility was designed to produce 25 tons per year of purified bulk active pharmaceutical ingredient (bulk API) from transgenic milk containing human serum albumin. For the comparison, all unit operations (see FIG. 6) were kept constant, and only the solution preparation and storage system and process utilities were modified to reflect the blending of buffers directly from acids, bases and additives. Moreover, due to the process of the current invention the facility (building) costs were reduced significantly, due to the reduction in space requirements by the elimination of many solution storage tanks and distribution piping. This also is reflected in the reduction in costs for the equipment needed for solution prep and CIP. There was also some reduction in the size and cost of the required water system. Overall, the estimated capital cost for the plant was reduced by $6.1 million (˜16%) through the introduction of the use of the methods of the invention.”

Although plentiful literature exists regarding the structure, function, and diseases associated with human serum albumin and alpha fetoprotein, the prior art does not disclose an efficient, automated, and continuous method of blending buffers and other solutions to process these proteins. With regard to alpha fetoprotein, U.S. Pat. No. 5,384,250 entitled “Expression and Purification of Cloned Alpha Fetoprotein,” explains a method for making human alpha fetoprotein in prokaryotic cells only. In addition, U.S. Pat. No. 5,206,153 entitled “Method of Producing Human Alpha-Fetoprotein and Product Produced Thereby” discloses a method to make human alpha fetoprotein whereby a DNA sequence for rat alpha fetoprotein is combined with the DNA for human alpha fetoprotein. These methods, however, do not yield a supply of human alpha fetoprotein by the use of the continuous, automated blending of buffers and other solutions.

As mentioned previously, this method may be employed to process human serum albumin and alpha fetoprotein for therapeutic applications. Serum albumin, the most well-known plasma protein, is responsible for a variety of physiological functions such as sustaining the osmotic pressure in the blood and transporting fatty acids and bilirubin (Peters 1995). Testing levels of serum albumin from feedstreams may be conducted to see if the subject has liver or kidney diseases or if an insufficient amount of protein is consumed by the blood. Decreased levels of serum albumin may signal such diseases as well as ascites, bums, glomerulonephritis, malabsorption syndrome, malnutrition, and nephritic syndromes.

In addition to measuring levels of serum albumin to detect disorders, synthesizing this protein is beneficial for therapeutic purposes. Albumin products are employed to maintain the plasma colloid oncotic pressure and to remedy severe edema by enabling intracavital and interstitial fluids to travel into the blood vessels. Albumin products may be administered to alleviate acute hypoproteinemia and pathological conditions stemming from chronic hypoproteinemia. Albumin products may be utilized to treat hypovolemic shock, severe bum injury, adult respiratory distress syndrome, ascites, liver failure, and pancreatitis. (Cochrane et al., 1998). Albumin may also be administered to remedy hyperbilirubinemia, hypoproteinemia, and nephrotic syndrome. (Vermeulen et al., 1995).

Alpha fetoprotein is another protein that may be processed for beneficial reasons. It is a protein assembled by the liver and yolk sac of a fetus. Throughout pregnancy, heightened levels may signal the following fetal abnormalities: spina bifida, anencephaly, omphalocele, tetralogy of Fallot, duodenal atresia, Turner's syndrome, and intrauterine death.

In addition to fetal diseases, monitoring increased levels of alpha fetoprotein may be useful in pinpointing cancers of the stomach, pancreas, biliary tract, testes, and ovaries, and recuperation from hepatitis.

According to an embodiment of the current invention when multiple or successive rounds of transgenic selection are utilized to generate a cell or cell line homozygous for more than one trait such a cell or cell line can be treated with compositions to lengthen the number of passes a given cell line can withstand in in vitro culture. Telomerase would be among such compounds.]

Accordingly, it is to be understood that the embodiments of the invention herein providing for an increased efficiency and speed in the production of chemical, biochemical, or biopharmaceutical processing are merely illustrative of the application of the principles of the invention.

It will be evident from the foregoing description that changes in the form, methods of use, and applications of the elements of the disclosed method for the improved buffer blending and development technology are novel and may be modified and/or resorted to without departing from the spirit of the invention, or the scope of the appended claims.

Prior Art Citations Incoprorated by Reference

1. Cochrane et al., Human Albumin Administration In Critically III Patients: Systematic Review Of Randomized Controlled Trials, BR MED J. (1998); 317:235-240.

2. Gibney M W, et al., Method of Beverage Blending and Carbonation, U.S. Pat. No. 5,552,171.

3. Jones, C, et al., Method for Blending Diverse Blowing Agents, U.S. Pat. No. 5,823,669.

4. Pak, Zinovy Petrovich—Chemical Compound, Or Soil Contaminated By Said Poisonous Agent and/or Toxic Chemical Compound, U.S. application Ser. No. 20020156336.

5. Patel M, et al., Apparatus for Blending Chemicals with a Reversible Multi-Speed Pump, U.S. Pat. No. 5,340,210.

6. Paul K D, et al., Method and Apparatus for Filling, Blending, and Withdrawing Solid Particulate Material From a Vessel, U.S. Pat. No. 4,907,892.

7. Phallen U, et al., Continuous Liquid Stream Digital Blending System, U.S. Pat. No. 6,186,193.

8. Platz G M, et al., Method and Apparatus for Super Critical Treatment of Liquids, U.S. Pat. No. 6,162,392.

9. Wilkins E, et al., Apparatus for Detecting Contamination in Food Products, U.S. Pat. No. 6,180,335.

10. Vermeulen L C, et al., Guidelines of or the Use of Albumin, Nonprotein Colloids, and Crystalloid Solutions, ARCH INTERN MED. (1995) 155:373-379.

What is claimed is:

1. A method for the production of aqueous pH buffered solutions or formulations comprising:
a) blending of water in a controlled manner; and
b) buffering acids and bases in solution at a controlled ratio to produce the desired final pH and buffer concentration from a source of constitutive acids and bases,

2. The method of claim 1 wherein any other other required ingredients of said buffered solution are added at a controlled ratio to produce the desired final concentration of each ingredient.

3. The method of claim 1 wherein said buffered solutions of the invention are used to process a biopharmaceutical.

4. The method of claim 1 wherein said biopharmaceutical is human serum albumin.

5. The method of claim 1 wherein the production of said buffered solutions is done continuously.

6. The method of claim 5 wherein a product feedstream is processed through simulated moving bed chromatography.

7. The method of claim 6 wherein a product feedstream is transgenic in origin.

8. The method of claim 7 wherein said transgenic product feedstream is milk.

9. The method of claim 6 wherein a product feedstream is derived from a cell culture broth.
